(19) United States
(12) Patent Application Publication
(10) Pub. No.: US 2003/0152639 A1
(43) Pub. Date: Aug. 14, 2003

(54) Novel wound healing composition not containing bovine-derived activating reagents

(76) Inventors: Calvin Britton, Little Rock, AR (US); Alex Dellinger, Little Rock, AR (US); Jim Limbird, Little Rock, AR (US); Carl Keller, Little Rock, AR (US); Charles Worden, Fayetteville, AR (US)

Correspondence Address: KEISLING & PIEPER PLC, Suite 217, One East Center Street, Fayetteville, AR, 72701, US

(21) Appl. No.: 10/323,861

(22) Filed: Dec. 17, 2002

Related U.S. Application Data
(63) Continuation-in-part of application No. 09/898,316, filed on Jul. 3, 2001, now abandoned.

(51) Int. Cl. A61K 35/14

(52) U.S. Cl. 424/529

(57) ABSTRACT

A wound care preparation free from bovine-derived activating agents is disclosed for use in wound care, for both topical wounds and surgical wounds. The preparation is isolated by first obtaining an amount of whole blood from the patient and treating the whole blood with one or more anti-clotting agents, subjecting the whole blood to a centrifugation process to obtain an amount of platelet-rich plasma, adding to the platelet-rich plasma an amount of anti-clotting neutralizing agent, and mixing the platelet-rich plasma with a structural matrix to increase viscosity of the preparation. In use, the viscous preparation can be applied directly to a wound or surgery incision and the viscous preparation may be mixed with other wound healing agents, growth matrices, or promoters such as anti-fungal agents, anti-biotic agents, and preservatives.

### CROSS-REFERENCE TO RELATED APPLICATIONS

This application claims priority and is a continuation-in-part of U.S. application Ser. No. 09/898,316, filed on Jul. 3, 2001.

### STATEMENT REGARDING FEDERALLY SPONSORED RESEARCH OR DEVELOPMENT

Not Applicable.

### REFERENCE TO A MICROFICHE APPENDIX

Not Applicable.

### BACKGROUND OF THE INVENTION

This invention relates generally to wound healing, and more particularly, to the use of platelet rich plasma free from bovine-derived reagents to facilitate accelerated healing for traumatized tissue.

Generally, it is well-known that a physical trauma to mammalian tissue sets in motion a chain of events to start the healing process. The first response by the body to tissue injury generally concerns an effort by the body to mitigate blood loss and to isolate the injury. Platelets are cellular elements that enter the wound site. The formation of a platelet plug, known as primary hemostasis at the site of injury, is coordinated by platelets and various factors and releasates. Secondary hemostasis concerns the formation of a fibrin clot, and the third step in the blood clotting process concerns the consolidation of the clot.

Serving as activators to trigger the platelet plug formation, collagen, thrombin, adenosine diphosphate, arachadonic acid, epinephrine, and platelet activating factor activate platelets by interacting with specific receptors on the platelet membrane. As part of this activation, calcium is mobilized, thereby instigating calcium-dependent processes which ultimately cause the release or secretion by the platelet the platelet granules. The platelet granules contain different biochemical mediators, which are categorized into different types; namely, dense granules, alpha granules and lysozomes. It is understood that the contents of dense granules serve to activate other platelets and to promote platelet aggregation and coagulation. It is also understood that the contents of alpha granules include fibrinogen, factor V, thrombospondin, alpha-antitrypsin, platelet derived angiogenesis factor (PDAF), platelet factor-4 (PF-4), transforming growth factor a (TGF-α), transforming growth factor β(TGF-β), endothelial cell growth factor, fibronectin, and platelet-derived growth factor (PDGF). It is generally understood that the foregoing alpha granule contents hold an important function as adhesive proteins, procoagulants, enzyme inhibitors, and growth factors, and that the release of the foregoing signal the local mesenchymal and epidermal cells to divide and increase collagen and glycosaminoglycan synthesis, among other actions. One physical manifestation of the release of activating factors is the overall contraction of cross-linked platelets and fibrin latticework to facilitate overall wound contracture. The generally understood benefit of wound contracture is to decrease wound volume and time to wound closure.

In addition to the platelet-driven wound healing pathways, the body also sets in motion other events, such as inflammation. The wound derives benefit from some level of inflammation because the inflammatory response increases blood flow, thereby allowing additional helpful cellular elements to reach the site of injury. Generally, after a blood clot is well-formed, secondary vasodilation and increased capillary permeability follow, producing acute inflammation. It is helpful to note that a prolonged inflammatory response brings with it certain unpleasant side-effects to the patient, the most prominent one being pain stemming from the localized edema, erythema, and heat inherent to inflammation.

The inflammatory response initially allows cells that are important to the immunological recovery of the wound to circulate into the wound, wherein the cells release agents such as cytokines, growth factors, interleukin-1, and tumor necrosis factor, to name only a few. Importantly, the composition of the wound changes during the inflammatory response. Whereas platelets and fibrin matrices were the initial primary component to form the blood clot, the incorporation of white blood cells such as macrophages and lymphocytes within the platelet and fibrin matrix creates a healing milieu such that collagen can be deposited and cross-linked, such that endothelial cells can regain an order and structure, and such that a scar can mature. It is important to understand that the platelet and fibrin matrix lays the scaffolding upon which a successful healing process can begin and continue. The present invention respects the importance of, and indeed emphasizes the importance of, the platelet and fibrin matrix to accelerate the healing response.

It is known in the field of therapy and wound care study to separate whole blood into various sub-components and to then study the individual components in an effort to define the processes that may accelerate wound healing. It is generally understood that the whole blood is separated by centrifugation such that at least three dominant components are isolated based on molecular weight. The three layers are understood to comprise red blood cells, platelet-poor plasma, and platelet-rich plasma. The platelet-poor plasma primarily contains predominantly plasma and fibrin or fibrin precursors. The platelet-rich plasma generally comprises platelets, white blood cells, fibrin, plasma, and plasma proteins.

To date, the prior art attempts to further identify or activate the individual components with bovine-derived reagents, to then purify the activated sub-components, and to arrive at a certain product such as a purified wound healing factor, or a fibrin glue product.

One field of this “blood component” study relates to using the platelet-poor plasma to obtain fibrin components, to treat the fibrin component with bovine-derived thrombin, and to arrive at a bioadhesive fibrin sealant. These bioadhesive compositions primarily contain the fibrin portion of the platelet-poor plasma layer whereby the activated fibrin is referred to as fibrin glue. In this approach, as in U.S. Pat. No. 5,631,019, the essential components for a fibrin glue are human fibrinogen (or a source of fibrinogen, such as a freeze-dried plasma protein concentrate of fibrinogen/factor XII/fibronectin) and an activating enzyme such as thrombin. Typically, this approach requires that prior to use, the plasma protein concentrates are solubilized in the presence of calcium chloride, thereby ultimately providing the necessary activation and crosslinking and stabilization of fibrin to become a tight mesh of fibrin glue. It is disclosed in the prior art that when this man-made fibrin glue is applied to the area of injury, that the fibrin clot adheres to the site of application to theoretically minimize bleeding, and promote wound sealing and potential wound healing.

As it is well understood in the prior art, thrombin is a common physiological instigator of blood clotting and serves as an integral element to the prior art's understanding and use of blood component activation. While thrombin is a naturally-occurring agent, the thrombin used in a clinical or laboratory setting is derived from a number of specialized sources such as reptilian venom; however, it is most commonly and economically derived from bovine sources. Traditionally, the bovine source of this thrombin is understood to receive a benefit because it is less likely to carry an HIM or hepatitis infectious risk; however, a minority of researchers acknowledge and caution that other bovine pathogens may be present. This is especially problematic in light of a recent and growing concern about bovine spongioform encephalitis disease, (“BSE”) and the inability to properly identify and screen against the transmissibility of certain Proteinaceous Infectious Units (PRIONs) such as BSE to humans. The growing concern of PRION transmission is logical because to date, it is unclear exactly the mechanism associated with the transmission thereof, and the relative size of the PRION allows the infectious unit to escape traditional means of detection.

One subset of blood component study relates to the autologous harvesting of the whole blood from a patient so as to minimize risk of infection, disease transmission, and immunological nonacceptance of the blood products. It is known in the prior art that an autologous source is more desirable when compared to an unknown donor source.

There are researchers and clinicians within this subset of autologous derived blood component study who regularly select the platelet-poor plasma layer of the blood and treat the platelet-poor plasma layer with platelet activating agents, such as thrombin and calcium salt, to produce a fibrin glue, discussed above. Generally, these practitioners treat the fibrin component with activating agents such as bovine-derived thrombin because of the economy associated with the bovine source.

And there are also researchers and clinicians within the subset of autologous derived blood component study who target the platelet-rich plasma. It is known in the prior art that certain practitioners in this narrow field first isolate the platelet-rich plasma layer, to then activate the platelets with bovine-derived thrombin, and then subject the activated platelets to further treatments such as washing and centrifugation to further isolate and purify the platelet releasates and then discard or ignore the remaining platelets, platelet structure, and other cellular structures originally contained in the platelet-rich plasma prior to applying the compound to the wound. The goal supporting this second-step isolation technique is to obtain, purify, and isolate a releasate, such as platelet-derived growth factor (PDGF), and to apply the concentrated platelet releasate to the wound to give the wound a large stimulus of specific growth factor. As with the platelet-poor researchers, the platelet-rich researchers invariably add bovine-derived thrombin as an integral step in obtaining the platelet releasate. It is important to note that in the prior art, the platelets themselves are not preferentially or generally applied to the wound in many instances, and it is only the concentrated growth factor that is understood to confer a benefit.

For example, some researchers and clinicians who focus on the platelet-rich plasma turn their attention to one specific growth factor that is released from platelets. In U.S. Pat. No. 5,165,938, the disclosure of which is incorporated herein by reference as if set forth verbatim, the inventor discloses the application of concentrated platelet derived growth factor (“PDGF”) to a wound because PDGF has been shown to be a major mitogenic protein for use in wound healing. Numerous prior art methods of obtaining an effective amount of PDGF have been disclosed, and many comprise platelet extracts or purified PDGF induction of either cell multiplication or DNA synthesis in cultured smooth muscle cells, fibroblasts and glial cells. Use of recombinant sources of PDGF is also addressed in the prior art. It is understood that PDGF is therapeutic for the treatment of injuries which require the proliferation of fibroblasts or smooth muscle cells to heal. One drawback to using PDGF is that the PDGF is sensitive to proteolysis and may be unstable in topical gel applications. Again, the philosophy supporting the PDGF isolation approach relates to the specific selection or isolation of but one of the many blood components or growth factors available for study.

To date, none of the prior art seems to address or focus on the looming risk of PRION transmission by virtue of activating with, or adding to, the platelets bovine-derived thrombin products or bovine-derived collagen products. Indeed, it has not been the object of the prior art to integrate the ability to avoid the use of bovine-derived components to address the concern of PRION or other xenographic disease transmission.

To date, the prior art seems to focus attention on an incremental and elemental analysis of only certain platelet release growth factors, thereby potentially ignoring the larger, more important and fundamental principles supporting wound healing and wound care. The present invention contemplates not only the individual components associated with certain steps in the overall wound healing, but also anticipates the requirement for proper structure, latticework, framework, and sequence within which the secondary inflammation response relates to the blood clot originally formed by the platelets and the corresponding releasates.

While the prior art activates, isolates and employs potentially unstable growth factors and independently applies said activated factors directly to the wound, the present invention contemplates the application of an autologous derived platelet rich plasma concentrate directly to the wound surface, thereby requiring the platelet-rich plasma concentrate to interface directly at the site of injury. It is essential in the present invention to note that it is the injured tissue that begins or initiates a sustainable and natural physiologic activation of the plasma-rich plasma concentrate, not the external and artificial activation with bovine-derived products in the prior art. Therefore, it has been a long-felt need to step back from an elemental factor-by-factor analysis and application of single or multiple growth factor elements. Instead, the present invention encompasses a broader and more comprehensive approach to wound care whereby a large majority of the necessary wound healing elements and scaffolding such as platelets, platelet cell membranes, fibrin, and white blood cells are provided in a concentrated form. The present invention represents a departure from the present trend since it is premised on the understanding that the collection of many wound healing elements serves a higher purpose when compared to the individual, incremental approach. Indeed, the present invention contemplates a synergistic effect of all components. Surprisingly, the present invention actually minimizes inflammation and the corresponding pain and swelling incident to a prolonged inflammatory response. The present invention is a more physiologically relevant regimen whereby a treated patient is not subjected to simply the immediate benefit of exogenous, artificial, and pre-activated autologous platelet derived wound care agents. Instead, the patient will enjoy a sustainable and directly acting reservoir effect incident to the auto-activation of platelet rich plasma. The present invention also contemplates the use of a carrier substrate in connection with the auto-activated platelet-rich plasma concentrate. The carrier substrate, in combination with the platelet-rich plasma concentrate, serves as a malleable structural matrix. This structural matrix, and the addition of potential preservatives and/or additives thereto, better enables the user to store, apply, handle, or manipulate the size of the tissue graft to a particular wound size and better enables the patient to go longer periods of time between bandage changes.

In short, providing the wound with more complete and essential healing elements in high concentrations will be more beneficial to the wound environment and will produce a greater likelihood of healing success when compared to the prior elemental approach found in the prior art. Moreover, because the relevant and necessary components are present, the presence of all essential healing elements will tend to decrease the inflammatory response, and accordingly, will tend to decrease the pain associated with the inflammation.

Admittedly, other researchers in the prior art do work with platelets in general and do not necessarily sub-purify platelet releasates, but these platelet researchers still rely on the use of bovine-derived activating agents as part of the overall goal in facilitating wound care. Therefore, it has also been a long-felt need to depart from the traditional bovine-derived activating agents, especially in view of the increasing threat of PRION transmission.

From a wound care bandage perspective, the blood component study prior art seemingly applies an amount of purified factors such as PDGF to the wound and simply covers it with a traditional gauze bandage. In surgical situations, a bandage is not available, and the purified factors are merely applied to the surface of the surgical incision.

In contrast to the prior art, the structural matrix substrate used in the present invention enables the user to properly conform the platelet-rich concentrate directly to the wound in a jelly-like vehicle. The present invention contemplates that the structural matrix may be selected from a group consisting of maltodextrin, hydroxy-ethyl cellulose, carrageenan, chitosan, oxidized regenerated cellulose, hydroxyethyl starch, hyaluronic acid, calcium alginate, methylcellulose, glycerin, but is not limited necessarily to the above-recited materials. Instead, the common feature of the above-described group of substrates is the ability to create a gelatinous consistency ranging from semi-liquid to semi-solid when mixed with unactivated platelet-rich plasma.

From a topical bandage perspective, the prior art seemingly relies upon traditional gauze bandages with a variety of different coatings. Moreover, the bandages are generally static and require frequent changes since another purpose of traditional bandages is to absorb bleeding. When the traditional gauze bandages become saturated, which is frequent in many instances, the gauze must be changed.

However, in the present invention, the use of the structural matrix allows a more flexible application and may in some cases extend the time between bandage changes. For example, calcium alginate is understood in the field of wound treatment to have characteristics that tend to absorb liquid. Accordingly, the present invention anticipates that calcium alginate, when mixed with the platelet rich plasma in an unactivated state, will work a dual purpose, both as a structural matrix to assist in the delivery of the platelet rich plasma to the wound site, but will also work to absorb extra liquid associated with the wound site, thereby increasing time that the bandage can stay on the patient, thereby maximizing potential for healing. Increasing the time that the bandage will properly provide a healing milieu with all necessary components present, and with minimized periods of interruption by virtue of lessened bandage changes, is the goal of the present invention and is consistent with an overall wound care philosophy.

### SUMMARY OF THE INVENTION

It is therefore an object of the present invention to provide a combination of concentrated autologous platelet-rich plasma product that is therapeutic in wound healing but does not utilize bovine-derived thrombin or bovine-derived collagen.

It is still further an object of the present invention to provide a preparation of concentrated autologous platelet-rich plasma in a relatively stable manner, as opposed to one that is unstable and perishable in a matter of hours.

It is still further an object of the present invention to provide a preparation of concentrated autologous platelet-rich plasma in a clinical environment to permit patients who experience recurrent wounds such as diabetic ulcers, to benefit from autologous wound care.

It is still further an object of the present invention to provide a unique concentrated autologous platelet-rich plasma that provides a substantial amount of natural wound healing components to be applied directly to, and auto-activated by, the wound, thereby decreasing wound healing time.

It is still further an object of the present invention to provide a combination of autologous platelet rich plasma and biodegradable or biodegenerative structural matrix material, to be applied directly to the wound, thereby decreasing the frequency for changing bandages or the trauma associated with changing bandages.

It is still further an object of the present invention to provide a topical protective elastomeric bandage that gains function by protecting the autologous platelet rich plasma product and structural matrix, and to draw together the wound, thereby decreasing wound volume, wound tension, and wound healing time.

It is still further object of the present invention to provide a preparation of autologous platelet-rich plasma and structural matrix material in an unactivated state such that the platelets remain substantially unactivated, and thereby more stable and potent, until the preparation is applied directly to the wound.

It is still further an object of the present invention to provide a preparation of concentrated autologous platelet-rich plasma and structural matrix that provides the necessary lattice-work or scaffolding to stimulate natural cellular infiltration and wound repair.

It is still further an object of the present invention to provide a method of treating a wound with a biologically-inert bandage impregnated with an autologous platelet-rich plasma.

It is still further an object of the present invention to impregnate the structural matrix with additives which serve to decelerate the naturally-occurring proteolysis or breakdown; to minimize risk of infection; to provide necessary medicine, anti-oxidants or vitamins; to provide ancillary treatment against fungi; to provide drugs or anesthetic to minimize pain; or to provide agents that combat disease, uncontrolled cellular growth, or uncontrolled cellular decay, or other accelerants to wound repair.

Towards the fulfillment of these and other objects and advantages, the present method relates to a first step of isolating from the patient an amount of whole blood and subjecting the whole blood to treatment with an anti-coagulant agent, followed by a centrifugation process to obtain an amount of platelet-rich plasma.

The second step involves adding an effective amount of Calcium Chloride to neutralize the anti-coagulant. A variety of other additives may be preferentially added to the platelet-rich plasma.

The third step involves adding to the platelet-rich plasma a structural matrix to increase viscosity and to adsorb extra liquid secreted from the wound. The structural matrix is selected from a group consisting of maltodextrmn, hydroxy-ethyl cellulose, calcium alginate, methylcellulose, glycerin, chitosan, carageenan, hydroxyethyl starch, hyaluronic acid, oxidized regenerated cellulose, and other non-toxic biomaterials that increase the viscosity of the concentrated autologous platelet-rich plasma preparation yet do not materially adverse the healing process.

This third step further serves to increase the viscosity of the platelet-rich plasma, thereby creating a gelatinous-type product and affording a relatively uniform distribution of the platelet-rich plasma throughout the structural matrix.

As a fourth step, the gelatinous preparation is applied directly to the wound. Subsequent to application, the wound is sealed either by surrounding tissue in the case of a surgical application or is covered with a topical bandage in skin care situations whereby a topical bandage is applied to provide a baffler to minimize risk of infection or insult to the treating compound. The present invention contemplates the use of a variety of bandages, including bandages that tend to exert a contractive unidirectional or multi-directional force to the wound to assist in wound closure. The bandage contemplated herein may further comprise an covering having an adhesive element and a contractive element such that the bandage may selectively or inherently apply a force towards the middle of the bandage, thereby applying some element of force to assist in closing the wound. The bandage contemplated herein may further comprise a variety of adjustment means that facilitate an active application of at least minimal closing force to the open wound.

### DETAILED DESCRIPTION OF THE INVENTION

The first preferred embodiment represents a method and related wound care preparation wherein the first step comprises first isolating from the equine patient, 450 cc of whole blood using venipuncture. As part of this isolation, it is preferable to receive the whole blood in a container that is treated with an effective amount of anti-clotting agent such as sodium citrate. Using a Cell Saver 5 at 4500 revolutions per minute, the autologous whole blood is centrifuged and thereby separated into the three different components; the platelet-poor plasma, the platelet-rich plasma, and the red blood cells.

To initiate the second step, the technician would selectively obtain from the centrifuged whole blood an amount of platelet-rich plasma and next treat the amount by adding Calcium Chloride (CaCl) in an amount between 0.1 and 1.0 cc of a 10% solution per 10 mL of autologous platelet-rich plasma. Addition of the CaCl serves to reverse the action of the anti-coagulant added during venipuncture. While a variety of other additives may be preferentially supplied to the platelet-rich plasma, depending on whether other treatments are needed such as an antifungal medicine or antibiotic agent, in this first preferred embodiment, no additive was supplied to the autologous platelet-rich plasma.

The third step involves suspending the platelet-rich plasma by mixing it with a relative amount of glycerin based hydrogel. This structural matrix is ordinarily packaged in gel form, so it is necessary to properly identify and quantify approximately one-half Curasol and one-half platelet-rich plasma to achieve jelly-like consistency. The consistency of the autologous platelet-rich plasma and the Curasol structural matrix serves to increase the viscosity of the platelet rich plasma, thereby creating a gelatinous-type product. The viscosity is a matter of judgment and will vary from application to application, with some factors that go into the consideration including the location of the wound, the physical condition of the wound. As a fourth step, the gelatinous autologous platelet-rich plasma and structural matrix preparation is applied directly to the wound and a topical bandage sufficient to cover all margins of the wound is applied to provide a traditional covering to minimize risk of infection or insult to the treating compound.

To more specifically address the nature of additives that may be selectively or in combination supplied in the second step or in the third step, the present invention contemplates additives comprising antibiotic agents, antimicrobial agents, antipathogenic agents, tumoricidal agents, coral for use with bone-regenerative efforts in orthopedic procedures, antiparasitic agents, tumoristatic agents, enzyme inhibitor agents, minerals, immunological camouflage agents or “masking agents” (to minimize recipient rejection), neurotransmitters, glycoproteins, antiviral agents, steroidal anti-inflammatory agents, non-steroidal anti-inflammatory agents, anti-cancer agents, anti-histimine agents, immunomodulator agents, visual marker elements, radiolabel agents, radio-opaque agents, radioflourescent agents, polysaccharide agents, cell receptor binding agents, nucleic acids, polynucleotide agents, immunoglobulin complexes, anti-tissue damage agents, monoclonal and polyclonal antibodies, hormones, immunosuppressive agents in tissue donor applications, vitamins, prostaglandins, enzymes, salts and buffer agents, preservatives, vasodilators, nitric oxide or precursors thereof, anti-arrhythmic agents, cardotonic agents, anti-hypertensive agents, hypotensive diruetic agents, sedatives, central nervous system agents, antitubercular agents, post-cerebral embolism agents, anti-ulcer agents, liposomes, neuraleptic agents, and growth factors.

Other modifications, changes and substitutions are intended in the foregoing, and in some instances, some features of the invention will be employed without a corresponding use of the other features. For example, it is anticipated that one or more additives may be used in connection with the second step or in the third step, whereby the additive is sufficiently blended with the structural matrix and the platelet-rich unactivated plasma. Moreover, the additives may be introduced immediately prior to the wound and may, in some cases, not be mixed with the platelet-rich unactivated plasma.

### EXAMPLE I

A human patient having a five month history of a foot ulcer under his fourth metatarsal head right foot was not progressing towards resolution with treatment with topical antibiotic ointment. The ulcer remained macerated, with a hyperkeratotic border. Treatment comprised application of unactivated platelets obtained by the procedure comprising steps described substantially herein. In the second step of this example, the platelet rich plasma was treated with 0.5 cc of CaCl, and 10 cc of platelet rich plasma concentrate was suspended with a calcium alginate to form the structural matrix dressing. On day one of treatment the ulcer volume was 1024 mm³, with a length of 16 mm, a width of 16 mm, and a depth of 4 mm. On day eighteen, the wound had decreased in volume to 50 mm³, with a length of 5 mm, a width of 5 mm, and a depth of 2 mm. At approximately three weeks post treatment, the wound had resolved, with no remaining volume.

### EXAMPLE II

An equine patient presented with injury to the right leg, with depth of injury to bone structure. Using traditional treatment protocol, no resolution of the wound was observed. Approximately twenty-five days post injury, the horse was treated with unactivated platelet rich plasma mixed with one-half Cursol hydrogel to form the structural matrix and covered with dressing. Ten days post treatment, the wound made substantial progress towards recovery and was filled with granulation tissue throughout the entire wound bed. Six weeks post treatment, the wound continued to heal and improve.

### EXAMPLE III

After obtaining PRP it is combined with one part powdered Vitamin C and 3 parts Chitosan. After several minutes a golden colored gel is formed. The gel can be applied to the wound bed and remainder stored and refrigerated for at least 5-7 days (the viable life span of a platelet) and subsequently used.

Gel viscosity can be controlled by

1) adding more PRP to make the gel less viscous

2) adding less Vitamin C to decrease the acidity therefore decrease viscosity

3) adding more Vitamin C to increase acidity and therefore increase viscosity.

### EXAMPLE IV

After obtaining PRP it is combined with Calcium Aliginate and allowed to congeal. The gel can be applied to the wound bed and the remainder stored and refrigerated for at least 5-7 days (the viable life span of a platelet) and subsequently used.

Gel viscosity can be controlled by

1) adding more Calcium Alginate to increase viscosity

2) adding less Calcium Alginate to decrease viscosity

What is claimed is:

1. A preparation for use in treating damaged tissue, the isolation thereof comprising the steps of:
(a) isolating from the patient an amount of whole blood, treating said whole blood with an anti-coagulant agent, and subjecting said whole blood to a centrifugation process to obtain an amount of platelet-rich plasma;
(b) adding to the platelet-rich plasma an effective amount of anticoagulant neutralizing agent and fibrinolysis inhibitor; and
(c) suspending the platelet-rich plasma by mixing it with one or more structural matrices from a group consisting of maltodextrin powder, hydroxyethyl cellulose, calcium alginate, carageenan, hydroxyethyl starch, hyaluronic acid, regenerated oxidized cellulose, methyl cellulose, and/or glycerin, to increase viscosity of the platelet-rich plasma and to form a gelatinous preparation; and,
(d) subsequently storing said gelatinous preparation in an unactivated state.

2. The preparation of claim 1 wherein said preparation is activated by application upon a wound.

3. The preparation of claim 2 wherein:
step (b) further comprises adding to the platelet-rich plasma an effective amount of one or more of the following wound-care agents selected from the group consisting of antibiotic agents, antimicrobial agents, antipathogenic agents, tumoricidal agents, antiparasitic agents, tumoristatic agents, enzyme inhibitor agents, minerals, neurotransmitters, glycoproteins, antiviral agents, steroidal anti-inflammatory agents, non-steroidal anti-inflammatory agents, anti-cancer agents, anti-histimine agents, immunomodulator agents, visual marker elements, radiolabel agents, radio-opaque agents, radioflourescent agents, polysaccharide agents, growth matrices, masking agents, cell receptor binding agents, nucleic acids, polynucleotide agents, immunoglobulin complexes, anti-tissue damage agents, monoclonal and polyclonal antibodies, hormones, immunosuppressive agents in tissue donor applications, vitamins, prostaglandins, enzymes, salts and buffer agents, preservatives, vasodilators, nitric oxide or precursors thereof, anti-arrhythmic agents, cardotonic agents, anti-hypertensive agents, hypotensive diruetic agents, sedatives, central nervous system agents, antitubercular agents, post-cerebral embolism agents, anti-ulcer agents, liposomes, nerualeptic agents, and growth factors.

4. The preparation of claim 2 wherein:
step (c) further comprises adding to the platelet-rich plasma an effective amount of one or more of the following wound-care agents selected from the group consisting of antibiotic agents, antimicrobial agents, antipathogenic agents, tumoricidal agents, antiparasitic agents, tumoristatic agents, enzyme inhibitor agents, minerals, neurotransmitters, glycoproteins, antiviral agents, steroidal anti-inflammatory agents, growth matrices, masking agents, non-steroidal anti-inflammatory agents, anti-cancer agents, anti-histimine agents, immunomodulator agents, visual marker elements, radiolabel agents, radio-opaque agents, radioflourescent agents, polysaccharide agents, cell receptor binding agents, nucleic acids, polynucleotide agents, immunoglobulin complexes, anti-tissue damage agents, monoclonal and polyelonal antibodies, hormones, immunosuppressive agents in tissue donor applications, vitamins, prostaglandins, enzymes, salts and buffer agents, preservatives, vasodilators, nitric oxide or precursors thereof, anti-arrhythmic agents, cardotonic agents, anti-hypertensive agents, hypotensive diruetic agents, sedatives, central nervous system agents, antitubercular agents, post-cerebral embolism agents, anti-ulcer agents, liposomes, nerualeptic agents, and growth factors.

5. The preparation of claim 1 wherein said preparation is stored for a period exceeding one day.

6. The preparation of claim 5 wherein said period exceeds one week.

7. The preparation of claim 1 wherein said preparation is stored for a period less than the viable life of a platelet.

8. A method for treating damaged tissue with platelet-rich plasma not specifically activated prior to treatment, comprising:
(a) subjecting an amount of whole blood to a centrifugation process to obtain an amount of a platelet-rich plasma;
(b) adding to the platelet-rich plasma an effective amount of Calcium Chloride;
(c) mixing with the platelet-rich plasma one or more structural matrices to form a gelatinous preparation;
(d) storing said gelatinous preparation for a period of time; and,
(e) applying the preparation directly to the damaged tissue.

9. The method of claim 8 wherein said period of time exceeds one day.

10. The method of claim 8 wherein said period of time exceeds one week.

11. The method of claim 8 wherein said period of time is less than the life span of a platelet.
